(19) United States
(12) Patent Application Publication
(10) Pub. No.: US 2026/0069556 A1
(43) Pub. Date: Mar. 12, 2026

(54) Use of N-Chlorotaurine for Treatment and Prevention of Viral Infections

(71) Applicants: Markus Nagl, Axams (AT); Frederic P. Zotos, Cohasset, MA (US)

(72) Inventors: Markus Nagl, Axams (AT); Frederic P. Zotos, Cohasset, MA (US)

(21) Appl. No.: 19/317,758

(22) Filed: Sep. 3, 2025

Related U.S. Application Data
(63) Continuation-in-part of application No. 18/946,372, filed on Nov. 13, 2024.
(60) Provisional application No. 63/693,319, filed on Sep. 11, 2024.
(60) Provisional application No. 63/700,892, filed on Sep. 30, 2024.
(60) Provisional application No. 63/769,415, filed on Mar. 10, 2025.

(51) Int. Cl. A61K 31/185; A61K 9/00; A61P 31/16

(52) U.S. Cl. CPC A61K 31/185; A61K 9/0043; A61K 9/0078; A61P 31/16

(57) ABSTRACT

Methods for treatment and prevention of a viral infection in an animal, including a respiratory infection, like a coronavirus such as severe acute respiratory syndrome coronavirus 2 (SARS-CoV-2), Influenza A such as H1N1, H3N2, H5N1, or respiratory syncytial virus. The compositions and methods include a therapeutically effective amount of about a 1% solution of N-chlorotaurine in water. The methods include administering N-chlorotaurine by eye drop to the eyes, nasal spray to the nostrils, by oral spray to the throat, by nebulizer to the lungs, and by cannula to the mammary glands.

### CROSS-REFERENCE TO RELATED APPLICATIONS

This is a continuation-in-part of application Ser. No. 18/946,372 filed on Nov. 13, 2024, with title “Compositions and use of N-chlorotaurine for treatment and prevention of respiratory infections,” which is a continuation of application Ser. No. 17/683,197 filed on Feb. 28, 2022, with title “Compositions and use of N-chlorotaurine for treatment and prevention of respiratory infections,” now U.S. Pat. No. 12,171,732, and naming Markus Nagl as inventor, the entire contents all of which are hereby incorporated herein by reference. This application claims priority to U.S. Provisional Applications Nos. 63/693,319 filed on Sep. 11, 2024, 63/700,892 filed on Sep. 30, 2024, and 63/769,415 filed on Mar. 10, 2025, the entire contents all of which are hereby incorporated by reference.

### FIELD

This invention relates to antimicrobial, anti-infective and antiseptic methods for the treatment and prevention of infections. More particularly, this invention relates to the use of N-chlorotaurine methods for the treatment and prevention of viral infections, especially coronavirus, influenza, and respiratory syncytial virus (RSV).

### BACKGROUND

Infections are a permanent problem in medicine. Airway infections including bronchitis and pneumonia are mostly caused by viruses and bacteria, but in the last decades also fungal infections have been increasing mainly due to immunosuppressive drugs which are used to treat cancer.

Many antibiotics are available to treat bacterial infections, and fewer drugs to treat viral and fungal infections. A problem with antibiotics, particularly with antiviral and antifungal drugs, is antimicrobial resistance. Some bacteria contain multidrug resistance genes and can be treated successfully only with a few available antibiotics. Recently, such genes have been found in fungi too, and a significant number of immunosuppressed patients die from fungal infections which are acquired through the airway route. To date, only a few viruses can be treated with specific antiviral agents.

Antiseptics are active against a broad-spectrum of pathogens usually including all mentioned classes of pathogens. If antiseptics are used in sufficient concentrations, acquired resistance (i.e., resistance which develops during repeated application of the same substance) does not occur. This is due to the unspecific reaction mechanism of most antiseptics, and is an advantage above antibiotics. However, antiseptics are much more toxic than antibiotics so that they cannot be applied systemically (i.e., intravenously, orally, intra-arterially). Only topical application to skin and mucous membranes is possible. There are delicate locations where not all antiseptics can be applied, e.g., the eye, body cavities such as paranasal sinuses, and the urinary bladder.

Special areas are the deeper airways, particularly the bronchopulmonary system, consisting of bronchi, bronchioli, and alveoli. Antiseptics are not used upon inhalation since significant concentrations are not tolerated because of irritative or toxic reactions, or considerable systemic resorption (e.g., alcohols, iodine). For instance, chloramine T has been shown to cause asthmatic and inflammatory symptoms after inhalative application.

Therefore, new compositions are needed for the treatment of infections of the bronchopulmonary system which can be applied topically, have sufficient activity against the causative pathogens, do not induce resistance of the pathogens, and are well tolerated upon inhalation.

Recently, the coronavirus disease 2019 (COVID-19) pandemic has been the major challenge for human health in this 21st century. The COVID-19 pandemic is caused by SARS-CoV-2. The pandemic is affecting individuals, populations, and health systems far beyond infection. The virus might persist globally and become a prolonged or permanent threat. The race for a cure is a global effort and different approaches have been proposed and are currently studied.

Another major public concern is posed by influenza viruses, which annually cause 3-5 million cases of severe illness and about 290,000 to 650,000 of death worldwide. Protection by the yearly influenza virus vaccine is unsatisfactory due to antigenic shift and mismatch, and resistance against existing antiviral drugs develops rapidly. Most influenza viruses are susceptible to human antiviral drugs, but the use of human antiviral drugs in animals is prohibited by government regulators to prevent the emergence of drug-resistant influenza viruses. Therefore, new tools to combat influenza viruses are urgently needed.

One less known intervention is inhalation therapy with antiviral agents. A first advantage is direct delivery of a high concentration of the medication to the lung, where the virus causes most of the severe problems. Furthermore, topically applied therapies that are not systemically distributed avoid interactions with systemic medications, which are frequently necessary in elderly or multimorbid patients who are particularly at risk for severe COVID-19 complications. An ideal inhaled drug should have broad-spectrum antimicrobial activity to cover not only SARS-COV-2, but also co-infections and superinfections with other respiratory viruses and microorganisms (bacteria and fungi).

Antiviral drugs are often specific to distinct viruses, but identifying the virus causing an infection requires logistic and diagnostic efforts, which in the case of SARS-COV-2 amounts to at best one to two days for a diagnosis. Such an ideal inhaled broad-spectrum drug mentioned above could be applied instantly regardless of the pathogen causing the respiratory illness and would thus eliminate the need for time-consuming diagnostics. A safe, well tolerated, endogenous substance with broad-spectrum activity against pathogens supported by anti-inflammatory properties may be a significant step forward for treatment of coronavirus, influenza, and other viral infections without the need of further diagnostics to discriminate between the infectious agents.

Influenza A viruses are masters of mutation and adaptation. It is of great concern that Highly Pathogenic Avian Influenza (HPAI) H5 (aka bird flu) has infected well over 150 different bird species and more than 50 mammalian species. Suitable medicines are needed to prevent and treat new waves of HPAI infection, especially in mammals, and ultimately to prevent human infection.

Recently, dairy cows have become the latest and most surprising victims in the lists of influenza A (H5N1) hosts. Infected cattle may be subclinical or clinical, and the main clinical signs are drastic decrease in milk production, changes in milk consistency, decreased feed uptake, dehydration and fever. The infection focuses on mastitis in the udders of lactating cows, and there are very high viral loads in the milk of infected cows. An effective medicine would decrease the viral load, inflammation, and other clinical symptoms to prevent or shorten the duration of the mastitis infection, thus allowing a quicker resumption of the milking of infected cows. An additional benefit would be if the medicine did not leave a residue like current antibiotics, which persist in the cows system for days and necessitate the discard of milk containing an antibiotic residue.

Importantly, there are concerns that cattle, once considered a resilient host, may act as a novel influenza A host in the future and after further adaptation of H5N1. This zoonotic virus has passed from cows to both poultry and humans, and also thereafter from poultry to humans. The risk of occupational exposure and disease spread exists amongst dairy and poultry farm workers and veterinarians. This is a zoonotic virus of pandemic potential, and there exists a need to mitigate risk and prevent the transmission of H5N1 among both people and animals.

HPAI is one of the most devastating avian viral diseases. The infection caused by HPAI induces an acute disease with high mortality reaching 100% in chickens. HPAI vaccination among chickens and domestic ducks has been implemented as a preventative measure and infection control in some countries. However, since the vaccines must be frequently updated due to antigenic changes of the viruses occurring in each epidemic, the vaccine may also lead to the silent spread of HPAI with asymptomatic infections, so vaccination against HPAI is prohibited for use in poultry in many countries. Furthermore, most U.S. trading partners will not accept exports of chickens from countries that allow vaccinations of chickens for bird flu due to concerns that vaccines can mask the presence of the H5N1 virus.

Therefore, highly sufficient and well tolerated medications for therapy and prophylaxis of infections, including COVID-19 and influenza viruses, are urgently needed.

### SUMMARY

In one aspect of the invention, there is provided a method of treating or preventing a viral infection in an animal, like a coronavirus, influenza A, and respiratory syncytial virus (RSV), the method comprising administering a therapeutically effective amount of N-chlorotaurine.

In various embodiments, the therapeutically effective amount of N-chlorotaurine is about a 0.1% to 2% solution of N-chlorotaurine in water.

In various embodiments, the coronavirus is severe acute respiratory syndrome coronavirus 2 (SARS-COV-2), or variants thereof, and the influenza A includes the H1N1, H3N2 and H5N1 influenza A virus strains.

In various embodiments, administering a therapeutically effective amount of N-chlorotaurine includes eye drop, aerosolized spray, inhalation, and intramammary instillation.

In one embodiment, the animal is a human, and the method comprises administering a therapeutically effective amount of N-chlorotaurine using an eye drop to the eyes, and an aerosolized spray to the nostrils/throat. The therapeutically effective amount of N-chlorotaurine is a dosage of about 0.1 ml to 0.2 ml to each of the eyes, about 0.1 ml to 0.3 ml to each of the nostrils, and about 0.3 ml to 0.6 ml to the throat, administered about 2 to 5 times per day.

In another embodiment, the animal includes a cow, goat, and sheep, and the instillation uses a cannula through a teat of an udder into a connected cavity of the mammary gland. The therapeutically effective amount of N-chlorotaurine is a dosage of about 50 ml to 100 ml per administration to the mammary gland, administered about 2 times per day.

In another embodiment, the animal is domesticated poultry including a chicken, turkey, duck, and goose, and the aerosolized spray uses a coarse spray over the whole bird (head and body). The therapeutically effective amount of N-chlorotaurine is a dosage of about 0.5 mg/Kg per administration to the whole bird, administered about 2 to 4 times per day.

It is understood that any aspect, feature, or embodiment of the invention can be combined with any other aspect, feature or embodiment of the invention, without departing from the scope of the invention. Other aspects and advantages of the invention will become apparent from the following description and claims.

### BRIEF DESCRIPTION OF THE FIGURES

FIG. 1A presents inactivation of SARS-Cov-2 (BAvPat1) by NCT. Virus suspension (SARS-COV-2 BavPat1) was incubated with 1.0% (55 mM) NCT, 0.1% (5.5 mM) NCT, 1.0% NCT with 5.0% peptone or PBS or 5.0% peptone for 5 min, 15 min, 30 min, or 60 min at 37° C., after which samples were diluted 1:1 in met/his solution for inactivation of NCT. Remaining infectious virus particles were determined using plaque titration. To control for inactivation of NCT by met/his, virus was added after dilution of 1.0% NCT with or without peptone in met/his. Mean values+SD of three to eight independent experiments in duplicates. The dotted line indicates the detection limit (0.84 log 10). Data were statistically analyzed using a two-way ANOVA including a Dunnett's multiple comparison test to PBS controls. *p<0.05, **p<0.01, ***p<0.001, ****p<0.0001. Of note, the inactivation of the virus by NCT was markedly enhanced in the presence of peptone.

FIG. 1B presents inactivation of SARS-Cov-2 (hCoV-19/Australia/VIC01/2020) by NCT. Virus suspension (SARS-COV-2 h CoV-19/Australia/VIC01/2020) was incubated with NCT or PBS or isopropanol (positive control) for 5 min, 10 min, 20 min, and 60 min at 37° C. and then diluted 1:1 in met/his for inactivation of NCT, followed by plaque titration. Mean values+SD of three independent experiments. Detection limit 3.11 log 10 (dotted line).

FIG. 1C presents inactivation of SARS-Cov-2 (SARS-COV-2, clinical isolate) by NCT. Virus suspension (SARS-COV-2, clinical isolate) was incubated with 1% NCT or PBS for 1 min, 5 min, 7 min, 10 min, 20 min, and 30 min at 37° C. After inactivation of NCT and serial dilution, aliquots were added to Vero/TMPRSS2/ACE2 cells for 1 h in 96-well plates. Cells were washed, incubated for further 9 h, and fixed for Immunostaining (c) or RT-qPCR (d). In immunostaining, infected cells were visible as red spots and counted using an ImmunoSpot S6 Ultra-V reader and CTL analyser BioSpot® 5.0 software. Mean values+SD of three independent experiments.

FIG. 1D presents inactivation of SARS-Cov-2 by NCT. After cell lysis and RNA extraction, one-step RT-qPCR assay was performed using the iTaq™ RT-PCR (BIO-RAD) kit and previously published primers and probes specific for detection of the SARS-COV-2 E Gene on a CFX96TM real-time system (BIO-RAD). Mean values±SD of genome copies of three independent experiments. Detection limit 2.10 log 10 RNA copies/ml (dotted line).

FIG. 1E presents inactivation of SARS-Cov-2 by NCT. Virus titration by TCID50. Mean values±SD of two independent experiments. Detection limit 1.50 log 10 (dotted line).

FIGS. 1F and 1G present inactivation of SARS-Cov-2 (variants, i.e. B.1.1.7 (Alpha) (FIG. 1F), B.1.351 (Beta) (FIG. 1G)) by NCT. Virus suspension (SARS-COV-2 variants, i.e. B.1.1.7 (Alpha) (FIG. 1F), B.1.351 (Beta) (FIG. 1G)) was incubated with 1% NCT or PBS for 1 min, 5 min, 10 min, 20 min, and 30 min at 37° C. After inactivation of NCT and serial dilution, aliquots were added to Vero/TMPRSS2/ACE2 cells for 1 h in 96-well plates. Cells were washed, incubated for further 9 h, and fixed for Immunostaining. Infected cells were visible as red spots and counted using an ImmunoSpot S6 Ultra-V reader and CTL analyser BioSpot® 5.0 software. Mean values±SD of three independent experiments.

FIG. 2A presents inactivation of Influenza A/Panama/2007/1999 (H3N2) virus by 1.0% (55 mM) and 0.1% (5.5 mM) NCT. Virus suspension was incubated with NCT or PBS for 5 min, 15 min, 30 min, or 60 min at 37° C., after which samples were diluted 1:1 in met/his solution for inactivation of NCT. Remaining infectious virus particles were determined using plaque titration. To control for inactivation of NCT by met/his, virus was added after dilution of 1.0% NCT in met/his in the inactivation control. Mean values±SD of five independent experiments. *p<0.05, **p<0.01, ***p<0.001, ****p<0.0001 versus PBS control. Detection limit 0.84 log 10 (dotted line).

FIG. 2B presents inactivation of Influenza A/California/Swine Origin Virus/2009 (H1N1pdm) virus by 0.1%, 0.5% and 1.0% NCT. Virus suspension was incubated with NCT in RPMI or plain RPMI for 1 min, 5 min, and 10 min at 22° C., after which samples were diluted 1:1 in met/his solution for inactivation of NCT. Remaining infectious virus particles were determined using plaque titration. Mean values±SD of four independent experiments. Detection limit 2.35 log 10 (dotted line).

FIG. 2C presents inactivation of Influenza A/Singapore/Hongkong/2339/2000 (H1N1) by 0.1% and 1.0% NCT. Test procedure and number of independent experiments as in FIG. 2B.

FIG. 2D presents inactivation of Influenza A/Singapore/Hongkong/2339/2000 (H1N1) by 0.1% and 1.0% NCT and by 0.1% NCT and 0.1% (5.5 mM) NCT plus 0.1% (18.7 mM) ammonium chloride compared. Test procedure and number of independent experiments as in FIG. 2B. Inactivation control in (FIG. 2D) consisting of 0.1% NCT plus 0.1% NH4Cl plus inactivator.

FIG. 3 presents inactivation of respiratory syncytial virus by NCT. Virus suspension was incubated with 1.0% or 0.1% NCT or PBS for 5 min, 10 min, 15 min, 30 min, or 60 min at 37° C., after which samples were diluted 1:1 in met/his solution for inactivation of NCT. Remaining infectious virus particles were determined using plaque titration. Mean values±SD of three independent experiments. Detection limit 1.90 log 10 (dotted line).

FIGS. 4A and 4B present chlorination of tyrosines, phenylalanines and tryptophan and oxidation of methionine of SARS-COV-2 spike protein by NCT. Spike protein was incubated for 30 min at 37° C. in 1% NCT and subjected to mass spectrometry. Positions of chlorinated and oxidized amino acids in the sequence are shown, with 18 tyrosines, two phenylalanines, and one tryptophan chlorinated and two methionines oxidized. No oxidation was found on cysteine.

FIG. 5 presents Chlorination of tyrosines and histidines and oxidation of methionines and cysteines of SARS-COV-2 3C-like proteinase by NCT. Proteinase was incubated for 30 min at 37° C. in 1% NCT and subjected to mass spectrometry. Positions of chlorinated and oxidized amino acids in the sequence are shown, with 3 tyrosines and two histidines chlorinated and seven methionines and four cysteines oxidized.

### DETAILED DESCRIPTION

The invention, in all of its various aspects and embodiments, comprises N-chlorotaurine (Cl—NH—CH2-CH2-SO3-) in compositions, devices, methods, and/or uses having antimicrobial properties in mammals, particularly humans.

N-chlorotaurine (Cl—NH—CH2-CH2-SO3-) is a small molecule that fulfils the criteria of broad-spectrum antimicrobial (virucidal, bactericidal, fungicidal, protozoocidal) and anti-inflammatory activity, and good tolerability upon inhalation. It is the product of activated human granulocytes and monocytes and belongs to the long-lived oxidants and chloramines formed by the myeloperoxidase via hypochlorous acid to combat invading pathogens. Moreover, N-chlorotaurine is thought to be involved in the control of inflammation by downregulating of nuclear factor kappaB activation, chemokines and proinflammatory cytokines such as tumor necrosis factor alpha, some prostaglandins and interleukins like IL-6.

The synthesis of the sodium salt of N-chlorotaurine (Cl—NH—CH2-CH2-SO3Na, NCT) is successful in the laboratory, which enables its development as an endogenous anti-infective and mild antiseptic in human medicine. Suitable salts can be prepared by known methods, including but not limited to the method described in German Patent Application 4041703, filed on Dec. 24, 1990, by Gottardi (incorporated by reference herein in its entirety).

As an active chlorine compound belonging to the class of chloramines, NCT has the broad-spectrum microbicidal activity without development of resistance against Gram-positive and Gram-negative bacteria including multi-resistant strains, yeasts and moulds, protozoa, and worm larvae. Broad-spectrum activity is found against adenoviruses, herpes viruses 1 and 2, human immunodeficiency virus, and is shown in vivo against adeno and herpes viruses in epidemic keratoconjunctivitis up to a phase II study as well as in herpes zoster in a case report, respectively. Activity against coxsackievirus A24 and enterovirus 70 is found by the NCT-derivative N,N-dichloro-dimethyltaurine in vitro.

Inhalation of NCT is being investigated and developed. NCT demonstrates enhanced bactericidal and fungicidal activity in the presence of lung epithelial cells. Tolerability of repeatedly inhaled NCT is confirmed in the normal lung and in a streptoccoccal inflammation model each in pigs, and in the normal lung of mice. In humans, tolerability is confirmed in a placebo-controlled phase I clinical study. Only minor and transient adverse effects were found, i.e. chlorine taste and occasional tickle in the throat. NCT is not distributed systemically, which explains the absence of systemic adverse effects.

In one embodiment of the invention, the alkali salt of N-Chlorotaurine (NCT), preferably the sodium salt, is used in the treatment and prophylaxis of a viral respiratory infection in a mammal, especially an infection of the lungs, the airways of the lower respiratory tract leading thereto, the upper respiratory tract, the nasal cavity, the oral cavity, or the throat. Examples of such viral infections include severe acute respiratory syndrome coronavirus 2 (SARS-COV-2), respiratory syncytial virus (RSV), influenza viruses (Influenza A or B virus), or derivatives thereof.

### In Vitro

NCT was incubated with SARS-COV-2, influenza A virus or RSV, followed by assessment of virus inactivation using various readouts. NCT at a clinically relevant concentration of 0.1%-1.0% demonstrated virucidal activity against SARS-COV-2 (SARS-COV-2 BavPat1, hCoV-19/Australia/VIC01/2020, clinical isolate 1.2 Innsbruck from early 2020, as well as the B.1.1.7 (Alpha) und B.1.351 (Beta) variants of concern, influenza A virus, and RSV (RSV long strain). Longer NCT-exposure periods were required to inactivate SARS-COV-2 than to inactivate influenza viruses or RSV. In the presence of organic matter, inactivation of viruses was even enhanced so that a significant reduction of plaque forming units and infected cells, respectively, could be observed already after 5 min with SARS-COV-2 by 1.0% NCT. Controls without NCT and specific inactivation controls showed full viral replication in all cases to warrant valid results. The multiplicity of target sites for NCT, which excludes the acquisition of distinct resistance mutations, was exemplarily demonstrated by mass spectrometry analysis of NCT-treated spike protein of SARS-COV-2. Detailed results are presented in the following paragraphs.

### Virucidal Activity of NCT Against SARS-COV-2

Inactivation of SARS-COV-2 wild type isolates from early 2020 was assessed by incubating stock virus with NCT for indicated time periods at 37° C. and then determining the remaining infectious particles using plaque assay or immunostaining as well as determining virus inactivation via RT-qPCR or TCID50. Exact incubation times of virus with NCT were ensured by adding met/his at the end of the incubation period, which inactivates NCT. All assays demonstrated a significant inactivation of SARS-COV-2 with slight differences according to the individual test method and strain used.

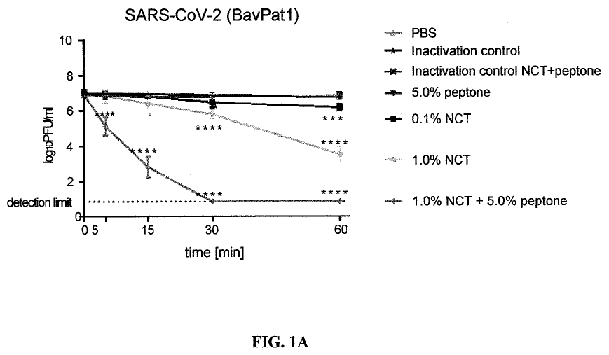

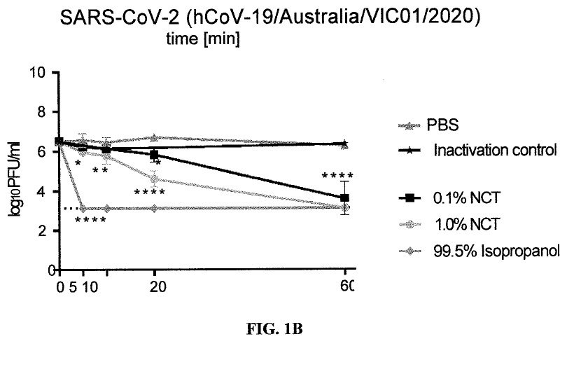

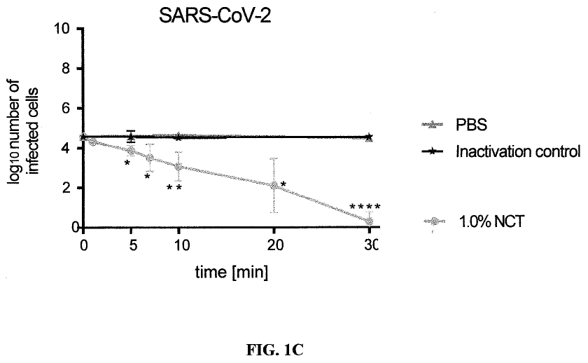

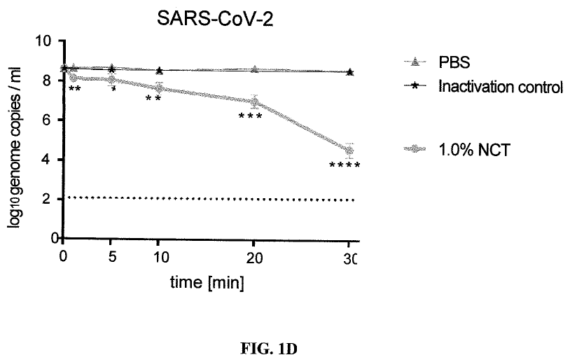

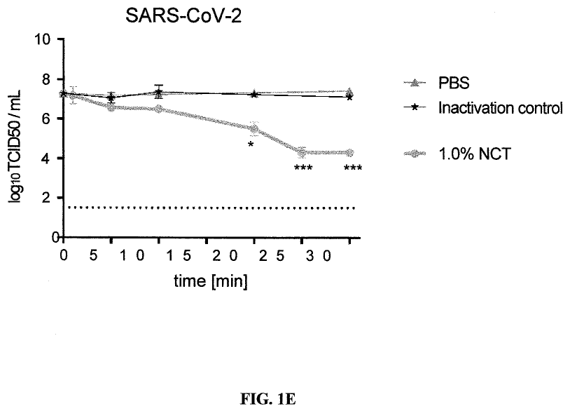

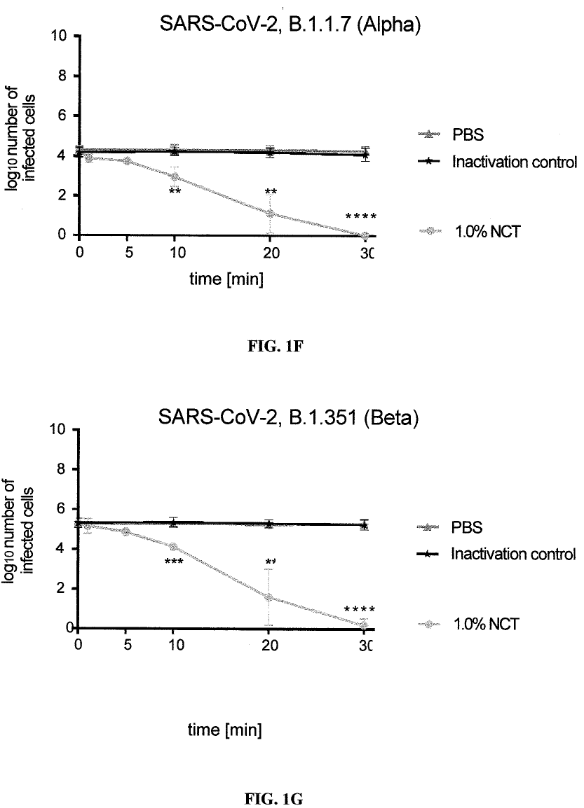

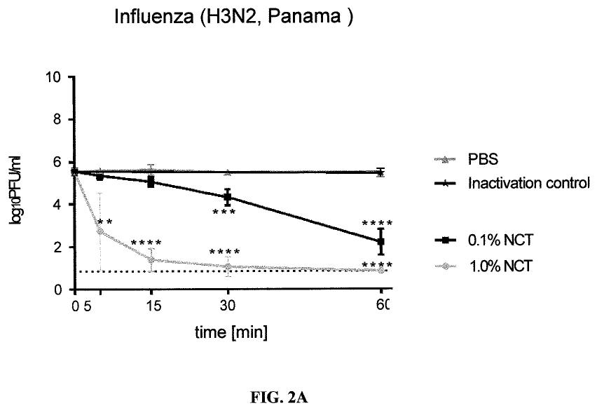

With plaque assay readout, a significant reduction in infectious particles was detected after 15 min of incubation, when incubating SARS-COV-2 with NCT in a buffered aqueous solution (FIG. 1A). The mild oxidizing activity of the test antiseptic may explain why it took as long as 15 min to reduce infectious particles. In the presence of Vero cells (FIG. 1B) or particularly 5.0% peptone (FIG. 1A), however, a significant reduction of infectious virus particles occurred already after 5 min of incubation with 1.0% NCT. This remarkable enhancement of activity for NCT by organic load is shown for viruses for the first time here and is explained most likely by transhalogenation.

Virus inactivation assays with immunostaining readout showed a 50% reduction of infected cells after 1 min (not significant, p=0.085), 20-80% reduction after 5 min (p=0.0102), 81-91% after 7 min (p<0.01), 81-97% after 10 min, 96-99% after 20 min, and >99% after 30 min (p<0.0001 for these values). A logarithmic scale with respective statistics is provided in FIG. 1C. The results found by RT-qPCR assay were similar with a highly significant reduction of genome copies (FIG. 1D). This was further confirmed by the TCID50 readout (FIG. 1E).

The representative isolates of SARS-COV-2 variants of concern B.1.1.7 and B.1.351 tested in the virus inactivation assay with immunostaining showed the same susceptibility to NCT than wild type isolates (FIGS. 1F and 1G). For the B.1.1.7 variant, the reduction of infected cells was 42-54% after 1 min (p=0.0109), 50-71% after 5 min (p<0.01), 62-99% after 10 min (p<0.01), 99-100% after 20 min (p<0.001), and 100% after 30 min (p<0.001). The values for the B.1.351 variant were 0-48% after 1 min (not significant, p=0.313), 54-72% after 5 min (p<0.01), 82-97% after 10 min (p<0.001), 99-100% after 20 min (p<0.001), and 100% after 30 min (p<0.001).

The antiviral activity was concentration-dependent. Inactivation controls demonstrated full inactivation of 1.0% NCT by 1.0% methionine/1.0% histidine (met/his). This was valid for all tests and viruses in this study. Absence of cytotoxicity of inactivated NCT to the inoculated cell culture was proved by MTT testing with values of MTT reduction of 94.1±8.5 (0.1% NCT plus 0.1% met/his) and 100.3±5.6 (PBS control) (p=0.12 by Student's unpaired t-test).

### Virucidal Activity of NCT Against Influenza Viruses

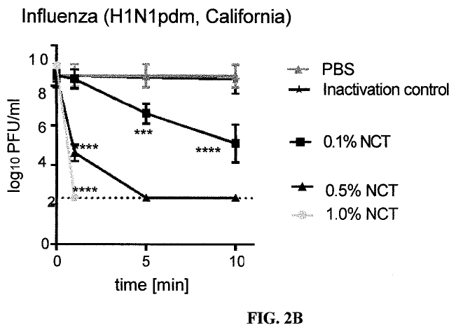

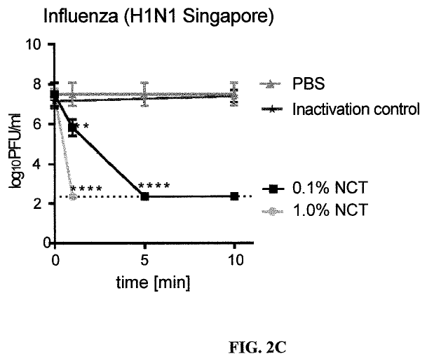

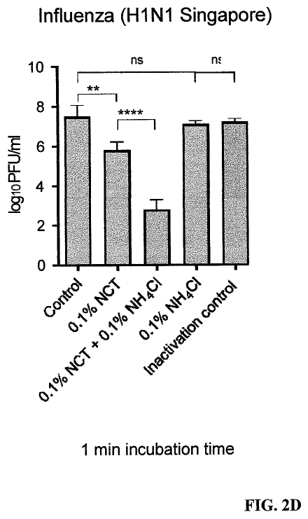

Inactivation of influenza viruses was assessed like inactivation of SARS-COV-2 by incubating stock virus with NCT for indicated time periods at 37° C. and then determining the remaining infectious particles using plaque assay. Virus inactivation as determined by plaque assay readout demonstrated an even faster inactivation of influenza viruses by NCT compared to SARS-COV-2. All tested virus strains were inactivated rapidly with a 2 log 10 reduction of the H3N2 virus within 5 min (FIG. 2A) and a 6 log 10 reduction of H1N1 and H1N1pdm viruses within 1 min by 1.0% NCT (FIGS. 2B and 2C). In general, H1N1 and H1N1pdm viruses were more susceptible than the H3N2 virus. Addition of ammonium chloride (NH4Cl) to NCT significantly enhanced its activity against influenza viruses (FIG. 2D). Ammonium chloride alone and the inactivation control with 0.1% NCT plus 0.1% ammonium chloride showed no antiviral effect at least up to 10 min incubation time.

### Virucidal Activity of NCT Against RSV

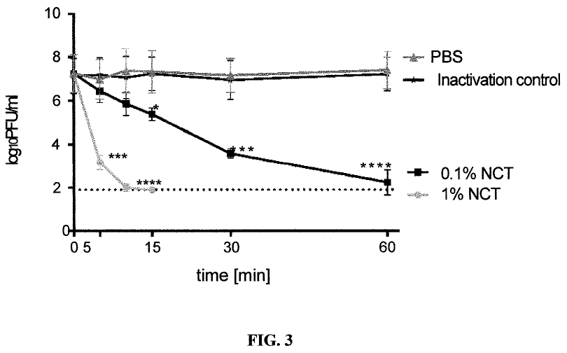

Inactivation of RSV was assessed as described for SARS-COV-2 and influenza A viruses. As with SARS-COV-2 and influenza A virus, inactivation of RSV as determined by plaque assay readout demonstrated a significant reduction of PFU/ml by NCT compared to mock treated controls. The incubation of RSV with 1.0% NCT resulted in a rapid drop of infectious virus titre with 4 log 10 decrease within 5 minutes (FIG. 3). Almost no detectable amount of infectious RSV was measurable after 15 minutes. In the presence of 0.1% NCT, RSV titres dropped in a time- and concentration-dependent manner reaching significant titre reduction after 15 minutes (FIG. 3).

### Structural Changes of Coronavirus SARS-COV-2 Spike Protein by NCT Evaluated by Mass Spectrometry

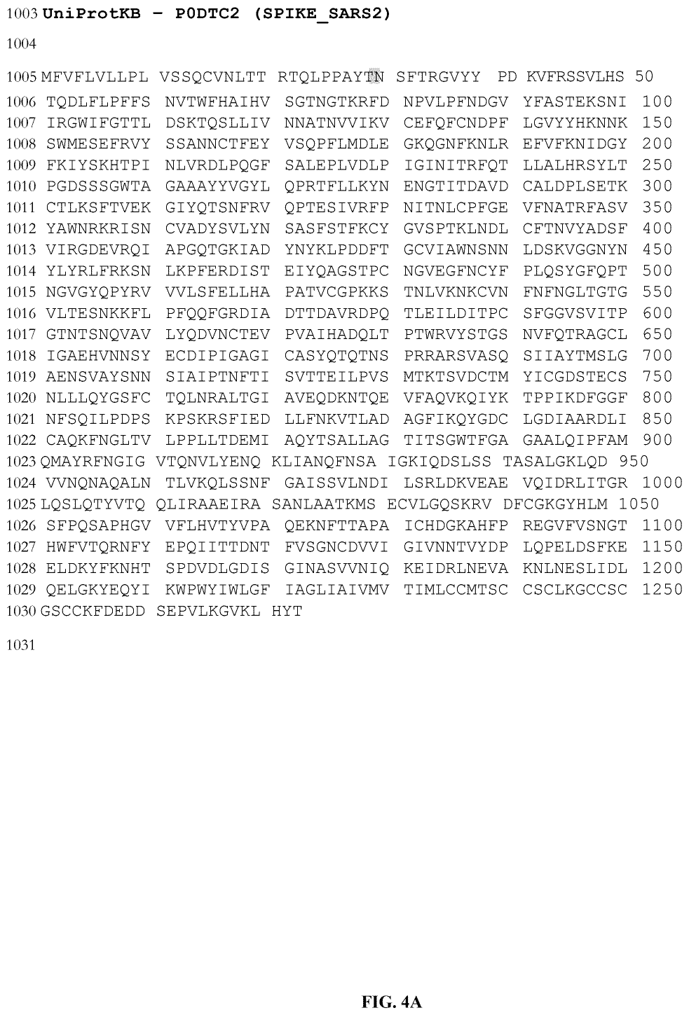

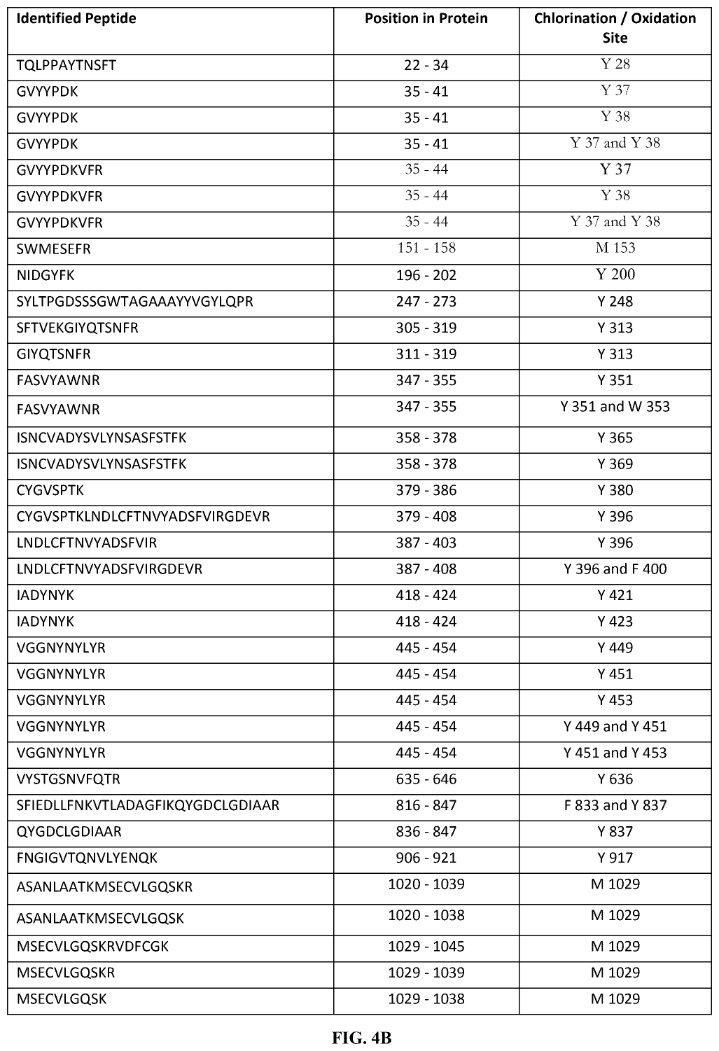

Multiple sites of oxidative attack by NCT could be demonstrated by mass spectrometry. Chlorination of aromatic amino acids was found, in detail of 18 tyrosines, two phenylalanines, and one tryptophan. Two methionines were oxidized. The exact positions of the chlorinated and oxidized amino acids are illustrated in FIG. 4. No oxidation of cysteine could be detected.

### Structural Changes of Coronavirus 3C-Like Proteinase by NCT Evaluated by Mass Spectrometry

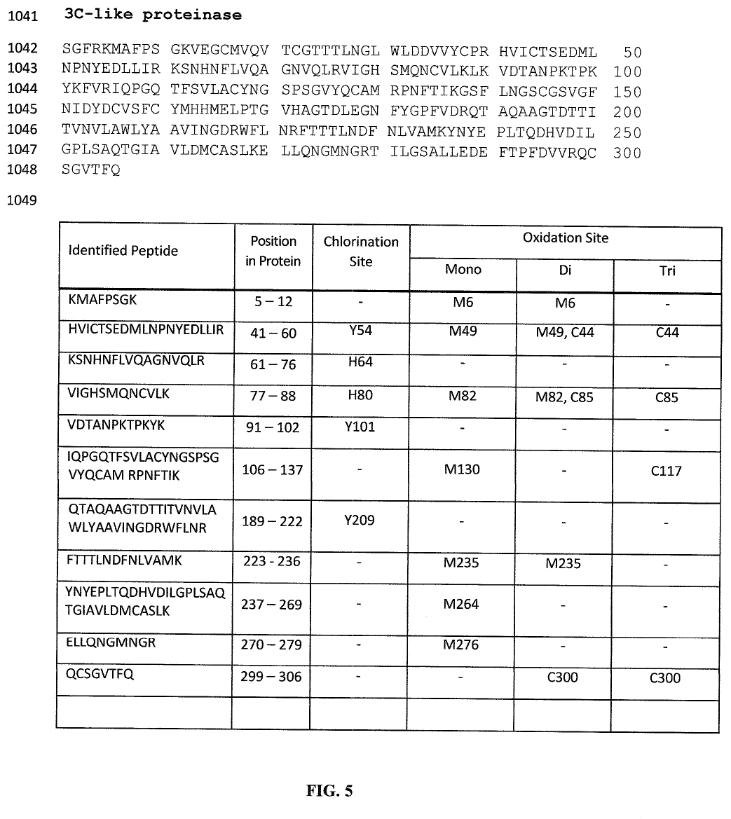

As with spike protein, multiple sites of oxidative attack by NCT could be demonstrated. Chlorination of aromatic amino acids was found, in detail of 3 tyrosines, and two histidines. Seven methionines and four cysteines were oxidized. The exact positions of the chlorinated and oxidized amino acids are illustrated in FIG. 5.

Discussion NCT has clear virucidal activity against three enveloped RNA viruses highly relevant for infections of the bronchopulmonary system. Depending on the NCT-concentration and test conditions, a rapid reduction of the number of infectious virus particles by several powers of ten within 1-10 minutes is achieved. Influenza A viruses of pre-pandemic and pandemic H1N1 subtype (H1N1 and HIN1pdm) were the most sensitive ones with reduction to the detection limit by 1.0% NCT within 1 min, followed by RSV, influenza (H3N2), and SARS-CoV-2. These differences can be explained by individual dynamics of oxidation and chlorination of proteins of the viral surface, and of penetration of NCT and attack on the viral nucleocapsid proteins. All these target sites have been shown with the NCT analogue N,N-dichloro-2,2-dimethyltaurine in adenovirus type 5. Thereby, chlorination of the surface proteins is the first step, which can be assumed to impact their function and therefore the attachment of viruses to body cells. The multiple sites of chlorination of tyrosine, phenylalanine, and tryptophan and oxidation of methionine by NCT in the spike protein found in the present study clearly confirm this principle in SARS-COV-2, too. Further targets are generally mainly thio groups and amino groups. Actually, we found oxidation of cysteine and methionine in the 3C-like proteinase of the virus besides chlorination of tyrosine and histidine, which underlines the attack of NCT at multiple proteins. Despite the occurrence of 40 cysteines in the spike protein (FIG. 4), no oxidation of them by NCT could be detected. A realistic explanation may be that they cannot be reached by NCT within the protein, which is underlined by their occurrence in part as disulfides (cystine).

Oxidation and chlorination of virulence factors of different pathogens by NCT and analogue chloramines with the consequence of their inactivation has been also shown for shigatoxin of Escherichia coli, several toxins of Staphylococcus aureus, aspartyl proteinases of Candida spp. and gliotoxin of Aspergillus fumigatus. This indicates that inactivation of key proteins of all kinds of pathogens is a central principle of the antimicrobial action of NCT and may underline such a function in innate immunity besides its anti-inflammatory one.

Accordingly, the activity of NCT against the SARS-COV-2 variants of concern was almost identical to that against the wild type. This result means that mutations in the protein sequence have no influence on the susceptibility of the virus to NCT. Similarly, multiresistance of bacteria and fungi against antibiotics and antifungals does not play a role for their susceptibility to NCT. As a further consequence, NCT has not only virucidal activity against enveloped viruses (herpes virus type 1 and 2, human immunodeficiency virus 1, and the viruses of the present study), but also non-enveloped ones. From the latter, a panel of adenoviruses has been tested mainly due to their importance in epidemic keratoconjunctivitis. Similar to other active halogen compounds and other antiseptics such as tensidic compounds, adenoviruses are slightly less sensitive to NCT than the enveloped viruses. Nevertheless, efficacy of NCT in vivo against adenoviruses in epidemic keratoconjunctivitis has been proven in the New Zealand White rabbit ocular model and in a phase II study in humans. Application of NCT had a curative effect in a patient suffering from therapy-refractory herpes zoster infection in the upper thoracic area.

It must be taken into account that organic substances are omnipresent in vivo (in all human body fluids and tissues), and therefore we performed a part of the inactivation assays in the presence of organic matter as well. The results of clearly show an enhancement of the virucidal activity of NCT in the presence of 5.0% peptone, which in the first view appears surprising since active chlorine compounds underlie a decrease of their oxidation capacity by chlorine-reducing substances of such organic load. With NCT as a low-reactive chloramine compound, however, transchlorination as one of the reaction mechanisms becomes important. Thereby, amongst others, monochloramine (NH2Cl) is formed in equilibrium from NCT and ammonium chloride.

Cl—HN—CH2-CH2-SO3-+NH4Cl↔H2N-CH2-CH2-SO3-+NH2Cl+H++Cl

NCT+ammonium chloride↔taurine+monochloramine

Monochloramine is more lipophilic than NCT and penetrates microorganisms more easily, which leads to enhanced inactivation by the reaction just mentioned. The stronger activity of NCT in the presence of fluids containing proteinaceous material is a general principle observed in different compositions, such as artificial sputum medium, different body fluids, peptone, and plasma for bacteria and fungi. In the present study, it has been confirmed for viruses for the first time, too. The discrepancy between the incubation time of 15 min and of 10 min or less needed for a significant viral reduction in buffer solution in different tests may be explained by the presence of 1.0% FCS in the tests and organic matter in the presence of Vero cells. In agreement with these results, enhancement of the bactericidal and fungicidal activity of NCT in the presence of different lung epithelial cells was observed recently.

Enhancement of antimicrobial and antiviral activity by organic material is of practical relevance for topical treatment of infections with NCT, for instance bronchopulmonary ones. The concentration of active chlorine after the end of an inhalation of 1.0% NCT decreases to traces within 1 min and vanishes completely after further 10 min. Inhalation for 10 min is feasible and well tolerated, and within this time an impact on SARS-COV-2 and on other viruses can be expected in vivo, too, but remains to be evaluated in respective clinical studies.

Also of practical relevance is the fact that NCT has broad-spectrum activity against viruses, including important representatives relevant for bronchopulmonary infections (SARS-CoV-2, influenza viruses, RSV). Of note, even different variants of the viruses with concerning phenotypes such as increased spread or virulence are similarly susceptible due to the unspecific, oxidizing and chlorinating mechanism of action of NCT. Topical treatment of all these virus infections by inhaled NCT without the necessity of a diagnosis of the specific virus at hand is conceivable and should urgently be investigated in clinical studies. Notably, the activity of NCT against bacteria and fungi, including multi-resistant ones, may prevent super- and secondary infections, which are a considerable problem in COVID-19 patients as well. In addition, the anti-inflammatory activity of NCT might have the potential to influence the aggressive inflammatory response by downregulating the “cytokine storm” and prevent airway damage in severe ill patients with SARS-COV-2 infection. Further advantages of NCT would be high safety and high tolerability by human tissue, absence of systemic absorption, of systemic adverse effects, of systemic interaction with other medications, and of resistance development because of the oxidizing and chlorinating mechanism of action.

Conclusion. NCT demonstrated rapid activity against SARS-COV-2, influenza A viruses, and RSV at a therapeutic concentration of 1.0% that can be safely inhaled. The molecular mechanism of action consists of oxidative attack at multiple sites of essential viral proteins, which excludes development of resistance and maintains virucidal activity against virus variants. The activity is enhanced by an organic environment, which is omnipresent in human body fluids and tissues in vivo. Clinical efficacy of NCT in viral bronchopulmonary infections are presented in the following examples herein.

### Treatment

NCT has been found to be useful in the treatment of COVID-19 (infection with SARS-CoV-2). Therefore, a particularly preferred embodiment of the invention is an alkali salt of N-Chlorotaurine, preferably NCT-Na, for use in the treatment of COVID-19 or infections with derivatives of SARS-COV-2.

NCT can be administered as a nasal/throat spray/rinse solution. The solution here consists of about 0.1% to about 5% NCT in a suitable solvent, a preferred embodiment is distilled water. A solution of about 1% NCT in distilled water is a particularly preferred embodiment.

In the first stages of disease, which is infection of the nose and throat, 1% NCT aqueous solution may be used as a nose spray (two to three puffs (about 0.1 ml/puff) per nostril during forced nasal inspiration) and/or throat spray (three to five puffs (about 0.1 ml/puff) in the back of the mouth to the throat). Alternatively, 1% NCT aqueous solution may be used as a nasal rinse (10 ml to 20 ml instilled for 30 seconds up to 1 minute) and/or throat gargling solution (10 ml to 20 ml for 30 seconds up to 1 minute). This may be repeated at least three times per day, and more often is even better (about 5 times a day) in the acute phase.

Early application is the best treatment strategy upon viral infections of the nose and/or throat region. Apply repeatedly and immediately upon the first itching in the nose or first scratching in the throat to inhibit the spread of the infection instantaneously or to attenuate the infection. If there is already a full infection established with marked symptoms, the application has demonstrated an attenuation of the symptoms. Treatment should be done for a few days until the pain and strong exudation goes away.

NCT must not be mixed with other substances in one solution. The patient should not eat or drink anything for at least 10 minutes, to avoid an impact on the activity of NCT since nutrition and fluids may inactivate NCT. There should be a period of at least 30 min to topical application of other substances to avoid mutual inactivation.

This treatment can markedly attenuate the course of viral flu-like infections. It has been observed to inhibit the occurrence of bronchitis in patients who always get bronchitis upon upper airway infections after a few days. The same inhibition has also been observed in COVID-19 patients, which means avoidance of the second stage of disease, which is development of pneumonia.

NCT-Na can also be administered as an inhalation solution. The solution here consists of about 0.1% to about 1% NCT-Na in a suitable solvent, a preferred embodiment is distilled water. A solution of about 1% NCT-Na in distilled water is a particularly preferred embodiment.

This solution is inhaled through a nebulizer (e.g. Pari Boy, Multisonic ultrasonic nebulizer, Akita Jet, Aero Eclipse II) until the symptoms disappear. Depending on the nebulizer, single doses of 3-10 ml of the solution can be administered, and about 3 ml is a preferred embodiment. These single doses are administered 1 to 10 times a day, and a preferred embodiment is 3 times a day.

Nebulizer: The size of the droplets must be sufficiently small in pneumonia (e.g. Covid-19), i.e. smaller than 5 micrometers. (Mass Median Aerodynamic Diameter MMAD<5 μm). If a deposition in the bronchi is desired, larger droplet size may be used.

Patients in the acute phase with pneumonia/bronchitis may inhale 2 to 3 times daily with 3 ml to 5 ml 1% NCT solution in the nebulizer for 10 min. Interval to possible other inhalations at least 15 min in case of 0.9% saline and 30 min in case of other inhaled medications.

May not mix with other drugs because of the oxidizing activity and mutual inactivation. Clean the reservoir of the nebulizer with water from other medications used previously.

### Prophylaxis

NCT may also be applied as nose/throat spray/rinse solution for prevention/prophylaxis of worsening or contagiousity to other persons. Even in case of low or no symptoms, 1% NCT aqueous solution may be used as a nose spray (two-three puffs (about 0.1 ml/puff) per nostril during forced nasal inspiration) and/or throat spray (three-four puffs (about 0.1 ml/puff) in the back of the mouth to the throat). Alternatively, 1% NCT aqueous solution may be used as a nasal rinse (10 ml to 20 ml instilled for 30 seconds up to 1 minute) and/or throat gargling solution (10 ml to 20 ml for 30 seconds up to 1 minute). This may be repeated at least two times per day or after close contact with other persons.

Of particular note, several cases have been observed where persons remained negative upon use of the 1% NCT nasal spray despite a close contact to COVID-19 positive persons, and even where other contact persons not using the 1% NCT nasal spray became positive.

### Viral Load

One main aim is to reduce the viral load in the upper airways. It is well known that ct values in the PCR above about 30 cycles lead to positivity in cell culture only in part. Above 35 cycles all cell cultures are negative. However, these numbers are variable since the PCRs of different labs are hardly well comparable.

NCT spray application leads to a decrease of infectiousity by decreasing of viral load compared to untreated controls, directly inactivating a partial number of the viruses, and attenuating a partial number of the viruses.

With bacteria and fungi, a short, sublethal incubation time of bacteria and fungi in NCT leads to a clear loss of their virulence in mice and moth larvae. At least one reason is immediate chlorination of the surface, which leads to damage of surface proteins and a lag of regrowth (postantibiotic/postantiseptic effect). The pathogens need the lag time to restore their surface and virulence factors to recover. This principle is the same for viruses: NCT will oxidize and chlorinate the surface proteins of SARS-COV-2, too, which will block attachment to cells. Also, the targets for the virus on the body cell surface are oxidized and chlorinated by NCT, so it has an immediate effect from both sides.

The invention is further explicated by means of the following examples.

Example 1. Treatment of COVD-19 patient with 1% NCT nasal spray, throat gargling solution, and inhalation.

Male patient in early 50s, without any underlying conditions. (Day 0): Symptoms onset: morning sweating and feeling sick, nasal secretion amplified, incipiating sore throat. Immediately began treatment with 1% NCT nasal spray (2-3 puffs (0.1 ml/puff) per nostril) and gargling solution (10 ml-20 ml for 30 sec-60 sec). Two hours later (about 9:30 AM), took smear of nose and throat in the morning, both positive in PCR; positive after 30-32 cycles PCR. Continued 1% NCT nasal spray and gargling solution (3× daily); inhaled 5 ml 1% NCT (2× daily) around noon and evening for 15 min. Sore throat did not occur, secretion became significantly lower. Redness of throat was very low. Feeling sick and weakness between 11:00 AM and 2:00 PM. (Day 1): Sweating and feeling sick in the morning, no more secretion, only from time to time still slight scratching of the throat with low cough irritation. Around 10:00 AM, continued 1% NCT nasal spray and gargling solution (3× daily); inhaled 5 ml 1% NCT (2× daily. Again temperature dropped in the morning. Did not lay down to rest during the day. (Day 2): Some night sweat; no respiratory problems, but muscle pain at the back; Continued 1% NCT nasal spray and gargling solution (3× daily); inhaled 5 ml 1% NCT (2× daily). Temperature normal. (Day 3): Some night sweat; no respiratory problems, muscle pain practically gone; temperature normal. Stopped 1% NCT therapy due to lack of symptoms. Throat swab made at 10:00 PM, in the morning both nostrils added. Result of the test two days later were positive after 25 cycles PCR. (Day 4): no complaints; (Day 5): no complaints; (Day 6): no discomfort: nose-throat smear at 11:00 AM. Test results on the same day: still positive after 27 cycles PCR. (Day 7): no complaints; (Day 8): no complaints; (Day 9): no complaints; (Day 10): no complaints; (Day 11): end of quarantine; no complaints; patient embarked on a winter cross-country mountain skiing tour. (Day 13): throat and nasal smear control PCR: weakly positive after 36.5 cycles (curve weak; max cycle number 45); (Day 19): throat and nasal smear control PCR: negative.

Discussion Patient began 1% NCT nasal spray and throat gargling therapy after the onset of symptoms on the morning of Day 0, but two-hours before taking first COVID-19 PCR test smear (30-32 cycles). Symptoms and NCT therapy stopped by day 3. However, the second PCR test smear was taken on the evening Day 3 (after the patient had stopped therapy) and was higher (25 cycles). A reasonable explanation for this result is arguably that the initial NCT therapy two-hours before the first PCR effectively reduced the viral load, whereas the stoppage of NCT therapy approximately 24 hours before the second PCR did not effectively reduce the viral load.

Example 2. Treatment of COVD-19 patient with 1% NCT nasal spray and throat gargling solution, and prophylaxis of family members with 1% NCT.

Male patient age in early 50s. His only symptom was a slight rhinitis. (Day 0): Swab with a PCR ct value of 14 cycles before NCT treatment on the first day. Start of 1% NCT therapy with nose spray (2-3 puffs (0.1 ml/puff) per nostril) and gargling solution (10 ml-20 ml for 30 sec-60 sec) about 6 hours later in the evening. (Day 1): Continuation of 1% NCT therapy (3× daily). Swab in the evening of the next day two hours after dosing: PCR ct value 20-22 cycles. Hardly any more symptoms. Continuation of 1% NCT therapy. (Day 2): Continuation of 1% NCT therapy. (Day 3): Continuation of 1% NCT therapy. (Day 4): Swab 3 days later in the evening of day 4: PCR ct value 31-34 cycles. No more symptoms. Continuation of 1% NCT therapy. (Day 5): Continuation of 1% NCT therapy on the whole next day. (Day 6): Swab in the morning of Day 6, which was 14 hours after the last dosing of NCT: PCR ct value equal 31-34 cycles. Stop of 1% NCT therapy due to this low value. No more symptoms, back to work after 10 days quarantine, swab negative.

Patient's wife and his three teenagers all residing in the same home also used 1% NCT nose and throat spray for 3 times daily as a prophylaxis, and they remained negative.

Example 3. Treatment of COVD-19 patient with 1% NCT nasal spray.

Male patient age 45. Previous Medical History of Mild Cardiomyopathy (10 years before) and Miocarditis (4 years before). (Day 0): early morning aches and muscle pain. Minor sore throat and nasal secretion. Feeling tired, generally ill and weak throughout the day. (Day 1): early morning aches, muscle pain, sweating. Minor sore throat and nasal secretion and cough. Feeling ill, tired, and weak throughout the day. Started to take Vitamin C, Zicam. Tested Positive for COVID-19 using rapid test. (Day 2): early morning aches, severe sweating (3 am) in morning. Increased cough and mucus secretion. 8 am feeling extremely ill, tired, pale, weak and experiencing upper back pain. 8:30 am-feat and palm sweats, uncontrollable chills and body shakes. Pulse drops to 40 bpm and admitted to ER. Given IV fluids, EKG, Chest X-Ray, and lab work completed. Lab work showed dehydration, IVF given, then released. (Day 3): experience loss of taste and smell, bad headaches, exhaustion. Very weak and tired. Continued back aches. (Day 4): bad headaches continue. Overall feeling of tiredness, nasal congestion, small cough, some mucus. (Day 5): bad headaches continue. Overall feeling of tiredness with periods of exhaustion. Nasal congestion with minor cough and mucus secretion. Began taking 1% NCT at 8 pm via nasal spray 2 puffs (0.1 ml/puff) in each nostril. Within 30 minutes nasal passages cleared and nasal secretion dried up. (Day 6): Continue general malaise throughout the day, headaches, overall feeling of weakness. Took NCT nasal spray 3× in each nostril at 8 am, 2 pm and 9 pm. (Day 7): Took NCT nasal spray 3× in each nostril at 8 am, 2 pm and 9 pm. Feeling better with more energy in the morning but experience tiredness by mid-day. Nasal passages and sinus clear, minor cough, no secretion. (Day 8): Took NCT nasal spray 3× in each nostril at 8 am, 2 pm and 9 pm. Feeling better with more energy in the morning but experience tiredness by mid-day. Nasal passages and clear, minor cough, no secretion. (Day 9): Took NCT nasal spray 3× in each nostril at 8 am, 2 pm and 9 pm. Feeling better with more energy in the morning but experience tiredness by mid-day. Nasal passages and clear, minor cough, no secretion. Night-time sweats. (Day 10): Took NCT nasal spray 3× in each nostril at 8 am, 2 pm and 9 pm. Feeling better with more energy with periods of tiredness. No cough or nasal secretion. (Day 11): Feeling better with more energy. Slight headaches come and go but overall better feeling. Stopped taking NCT. (Day 12): Feeling better with more energy. Slight headaches come and go but overall better feeling. (Day 13): Feeling better with more energy. Slight headaches come and go but overall better feeling.

Discussion Overall, it took patient full three weeks to fully recover from COVID-19. By week three headaches were not as bad, and generally experience more energy throughout the day. By the end of week three slowly began to start exercise again. Patient reported that he started feeling better within minutes of beginning NCT therapy, and he felt that this was the turning point towards his recovery.

Example 4. Treatment of COVD-19 patient with 1% NCT nasal/throat spray, and prophylaxis of spouse with 1% NCT.

Male patient age 32 years with previous history of smoking cigarettes (quit 1 year ago). (Day 0): tested positive for COVID-19 (rapid test). (Day 3): symptoms of stuffy nose and sore throat. (Day 4): Began taking 1% NCT therapy, i.e., nasal spray (3× (0.1 ml/puff) per nostril) and throat spray (5× puffs (0.1 ml/puff)) in the back of the mouth to the throat; dose time and symptoms: 11:00 AM—Sore throat, mucus 4:30 PM—Sore throat, less mucus 10:25 PM—Some dry mucus 2:15 AM—Less sore throat, dry mucus. (Day 5): dose time and symptoms: 12:05 PM—Sore throat, mostly clear nasal passages; 4:30 PM—Sore throat, minor mucus 2:00 AM—Minor symptoms (didn't write this one down, but patient thought he took a dose at this time so he included it). (Day 6): dose time and symptoms: 12:05 PM—Minor soreness in throat, mucus 8:05 PM—Mild sore throat and mild mucus 12:05 AM Mild sore throat and mild mucus. (Day 7): dose time and symptoms: 2:05 PM—Sore throat, minor mucus 5:15 PM—Minor soreness in throat 8:25 PM—Minor sore throat, minor mucus 12:43 AM—No symptoms. (Day 8): dose time and symptoms: 1:13 PM—Minor sore throat 7:17 PM—Minor sore throat 3:43 AM—No symptoms. (Day 9): dose time and symptoms: 12:30 PM—Minor mucus 4:48 PM—No symptoms 9:00 PM—No symptoms. (Day 10): dose time and symptoms: 10:57 PM—No symptoms.

Spouse, Female age 32. (Day 7 of husband's infection) dose time and symptoms: 2:10 PM—No symptoms 8:25 PM—No symptoms. (Day 8 of husband's infection) dose time and symptoms: 2:40 PM—No symptoms 11:30 PM—No symptoms. (Day 9 of husband's infection) dose time and symptoms: 11:10 AM—No symptoms 7:15 P. No symptoms.

Example 5. Treatment of Patients who suffer from COVID-19 with NCT solution for inhalation.

The effect of an inhalation solution of NCT-Na is tested in patients who suffer from COVID-19 (infection with SARS-COV-2). The solution consists of 1% NCT sodium in distilled water.

Eight hospitalized patients inhaled 3 ml of this solution 3 times a day for 1 week to 9 days via a nebulizer, and were then able to leave the hospital with survived illness and symptom-free or largely symptom-free.

Another 20 hospitalized patients suffering from COVID-19 were concurrently undergoing the same treatment, during which there was no progression of the disease and lung failure was avoided.

Example 6. Inactivation of HPAI Influenza A/Cattle/Texas/2024 (H5N1) virus by 0.1% (5.5 mM), 0.5% (27.5 mM), and 1.0% (55 mM) NCT.

Influenza A/Cattle/Texas/2024 (H5N1) virus suspension was incubated with NCT or PBS for 5 min, 15 min, 30 min, or 60 min at 37° C., after which samples were diluted 1:1 in met/his solution for inactivation of NCT. Remaining infectious virus particles were determined using plaque titration. Mean values±SD of three independent experiments. All assays demonstrated significant and rapid inactivation of the H5N1 virus particles by several powers of ten within 5-15 minutes. The antiviral activity was concentration-dependent. Inactivation controls demonstrated full inactivation of NCT by 1.0% methionine/1.0% histidine (met/his).

Example 7. Treatment of lactating cows which suffer from HPAI Influenza A (H5N1) viral mastitis with 1.0% NCT intramammary gland instillation.

The bulk milk tanks from a herd of 800 lactating dairy cows tested positive by PCR for HPAI H5N1 Bird Flu, and the herd was placed under quarantine. Seventy of the cows with clinical signs of mastitis were separated from the herd and placed into a hospital pen. These cows presented serious symptoms of acute clinical mastitis with low energy, their milk production was significantly off, they were not eating well, and they were lethargic.

Ten of the sickest cows in the hospital pen were treated twice daily (after the morning and evening milkings) for a period of five days with 1% NCT. Single doses of 100 ml per quarter were injected into all four quarters (for a total dose of 400 ml per cow) by intramammary instillation using 100 ml syringes with the appropriate adapter subsequent to milking. The cows appeared to tolerate the treatment well. After 4-5 days of NCT therapy, the cows appeared to be different cows. They were in much better shape, remarkably happier, produced more milk, and they just looked great (like “rock stars”). Their milk production (amount/quality) was significantly up, they were stronger, eating better, and they were recovering faster than the untreated cows. Their overall presentation was much improved when compared to the balance of the subset of the herd in the hospital pen.

Following the NCT treatment of the first ten cows, the remaining sixty cows in the hospital pen were treated in the same manner twice daily (after the morning and evening milkings) for a period of three days with 1% NCT. The condition of these sixty remaining cows similarly improved like the first group of ten NCT treated cows, and they no longer showed clinical signs of mastitis. Subsequently, all seventy cows were released from the hospital pen and rejoined the herd of 800 cows.

The forgoing examples are illustrative only, and not meant to limit the scope of the inventions claimed herein.

The invention is further explicated by means of the following prophetic examples.

Example 8. Treatment of lactating cows challenged by HPAI Influenza A/Cattle/Texas/2024 (H5N1) virus with 1.0% NCT intramammary gland instillation.

Four non-pregnant, lactating cows of approximately 3 years of age are examined clinically, and it is determined they are healthy and without infections of the udder. Solutions may be applied by known methods, including but not limited to the method described in Baker et al., “Experimental reproduction of viral replication and disease in dairy calves and lactating cows inoculated with highly pathogenic avian influenza H5N1 clade 2.3.4.4b,” bioRxiv preprint doi: https://doi.org/10.1101/2024.07.12.603337 (Jul. 13, 2024) (incorporated by reference herein in its entirety). The cows are inoculated with 1 ml of 1×10⁵ tissue culture infectious dose 50 (TCID50) Influenza A/Cattle/Texas/2024 (H5N1) virus by intramammary route in each of two quarters, front right and rear left, using a teat cannula. After instilling, the inoculum is moved upward into the teat sinus with gentle message.

Two cows each are treated twice daily (at 9:00 h and 15:00 h) started one hour after inoculation for 5 days in total with either 1% NCT (test) medication, or 0.9% saline (control) medication into each of the two H5N1 inoculated quarters, front right and rear left. Solutions may be applied by known methods, including but not limited to the method described in German Patent Application DE 10 2007 015 824, “Compositions and methods of treatment of mastitis,” by Gottardi (incorporated by reference herein in its entirety). Single doses of 100 ml per quarter are injected by intramammary instillation using 50 ml syringes with the appropriate adapter subsequent to disinfection of the teats with 70% ethanol.

Following inoculation, both cows treated with 0.9% saline show signs of mastitis with positive California Mastitis Test (CMT, ImmuCell, Portland, ME), and milk color and consistency changes begin 2 days post inoculation (DPI) and last until approximately 14 DPI, only in the inoculated quarters. Rumen motility starkly declines on 1 DPI and begins recovering after 7 DPI. Milk production steadily declines from 1-4 DPI, remains low until 10-12 DPI, and is 71-77% of pre-inoculation production at 23 DPI. The cows show signs of lethargy, reduced feed intake, self-resolving watery diarrhea or dry feces, and intermittent clear nasal discharge throughout the 24-day observation period.

Following inoculation, both cows treated with 1% NCT show no signs of mastitis with positive CMT, and with slight milk color and consistency changes beginning at 2 DPI and lasting until approximately 5 DPI, only in the inoculated quarters. Milk production declines slightly but significantly on 1 DPI, begins recovering after 5 DPI, and is 100% of pre-inoculation production at 10-12 DPI. The behavior of the cows does not change during the study. Food and water uptake is normal, and no abnormal vocalizing occurs. Daily clinical examination does not reveal any pathologic findings.

Example 9. Treatment of Influenza A patient with 1% NCT nasal/throat spray, and post exposure prophylaxis of household contacts.

Male patient age 65 years with underlying medical conditions of asthma, hypertension, obesity, and type 2 diabetes. (Day 0): Symptoms onset: develops fever, shortness of breath, diarrhea, nausea and general malaise. Tests positive for Influenza A (rapid test). Begins taking 1% NCT therapy (3× daily), i.e., nasal spray (3×(0.1 ml/puff) per nostril) and throat spray (5× puffs (0.1 ml/puff)) in the back of the mouth to the throat. (Day 1): Symptoms generally improve, with only slight fever and improved breathing. Continue 1% NCT therapy. (Day 2): Symptoms continue to improve, without fever and normal breathing. Continue 1% NCT therapy. (Day 3): Hardly any more symptoms. Continue 1% NCT therapy. (Day 4): No more symptoms. Stop taking NCT.

Patient's mother and brother (all residing in the same home) also use 1% NCT nasal/throat spray therapy (2-3× daily) as a prophylaxis for the duration, and they remain negative without developing any symptoms.

Example 10. Treatment of Influenza A patient with 1% NCT inhalation, and 1% NCT nasal/throat spray post exposure prophylaxis of household contacts.

Female patient age 96 years with underlying medical condition of hypertension. (Day 0): Symptoms onset: develops upper respiratory tract signs of bronchitis and pharyngitis, general malaise and myalgia. (Day 1): Symptoms generally deteriorate. (Day 2): Symptoms continue to deteriorate, and develops shortness of breath. (Day 3): Symptoms continue to deteriorate, and develops pneumonia. Tests positive for Influenza A (rapid test). Begins taking 1% NCT therapy, i.e., inhales 3 ml 1% NCT for 10 minutes (3× daily). Within minutes of each NCT therapy, breathing temporarily improves. (Days 4-5): Symptoms stabilize. Continue 1% NCT therapy. (Days 6-7): Symptoms generally improve, with improved breathing and less fatigue. Continue 1% NCT therapy. (Day 8): Symptoms continue to improve, with improved breathing. Continue 1% NCT therapy. (Day 9): Hardly any more symptoms except for cough. Continue 1% NCT therapy. (Day 10): No more symptoms. Stop taking NCT.

Patient's two sons (all residing in the same home) use 1% NCT nasal/throat spray therapy (2-3× daily) as a post exposure prophylaxis for the duration, and they remain negative without developing any symptoms.

Example 11. Treatment of RSV patient with 1% NCT inhalation, and 1% NCT nasal/throat spray post exposure prophylaxis of household contacts.

Male patient age 9 years with underlying medical condition of asthma. (Day 0): Symptoms onset: develops fever, runny nose, nasal congestion, cough, sneezing, wheezing and shortness of breath. (Day 1): Symptoms generally deteriorate. (Day 2): Symptoms continue to deteriorate. Tests positive for RSV (rapid test). Begins taking 1% NCT therapy, i.e., inhales 3 ml 1% NCT for 10 minutes (3× daily). Within minutes of each NCT therapy, breathing temporarily improves. (Day 3): Symptoms stabilize. Continue 1% NCT therapy. (Day 4): Symptoms generally improve, with only slight fever and improved breathing. Continue 1% NCT therapy. (Day 5): Symptoms continue to improve, with no fever and improved breathing. Continue 1% NCT therapy. (Day 6): Hardly any more symptoms except for cough. Continue 1% NCT therapy. (Day 7): No more symptoms. Stop taking NCT.

Patient's mother, father, younger sister and brother (all residing in the same home) use 1% NCT nasal/throat spray therapy (2-3× daily) as a post exposure prophylaxis for the duration, and they remain negative without developing any symptoms.

Example 12. Simultaneous and delayed treatment of chickens challenged by HPAI Influenza A/Cattle/Texas/2024 (H5N1) virus with NCT.

For all challenge experiments, 6-week old chickens (0.5 kg body weight) are used in this study. Two experiments are carried out to mimic the different field conditions of infection during an HPAI outbreak and subsequent treatment.

In the first experiment, chickens are divided into five groups of nine birds each (0.1%, 0.5%, 1.0% and 2.0% NCT, and 0.9% sodium chloride as placebo control). The chickens are infected intranasally with 100 μl of lethal Influenza A/Cattle/Texas/2024 (H5N1) virus. Treatment via eye drop by pipette (which makes its way into the respiratory tract via the lacrimal duct) with 20 μl aqueous NCT solution or 0.9% sodium chloride as placebo three times daily is immediately started after inoculation and ended after 5 days. NCT concentrations tested are 0.1%, 0.5%, 1.0% and 2.0%. Main parameters of evaluation are survival over time and viral load, and secondary ones are clinical parameters.

In chickens treated with placebo, all die from infection by 3 days post inoculation (dpi), while 0/9 to 1/9 chickens die in groups treated with 0.5%, 1.0% and 2.0% NCT (p<0.01 for each concentration versus saline). With 0.1% NCT, 4/9 chickens die (p=0.03 versus the higher NCT concentrations; p=0.0035 versus control) by 5 dpi. Although infection is established in chickens by inoculation with HPAI virus, viral excretion and clinical signs are predominantly suppressed by NCT activity at a concentration of 0.5% or higher, enabling the chickens to survive infection.

In the second experiment, the chickens are divided the same as in the first experiment. However, the treatment for all chickens starts 24 h after the infection when the first clinical signs such as depression and lack of appetite appear, which mimics the field situation of an HPAI outbreak. Drugs are then administered three times a day for 5 days.

All the chickens in the five groups (0.1%, 0.5%, 1.0% and 2.0% NCT, and control) succumb to the HPAI infection. However, in groups treated with 0.5%, 1.0% and 2.0% NCT, the chickens survive up to 5 dpi, and in the group treated with 0.1% NCT the chickens survive up to 4 dpi, whereas in the placebo group all the chickens die by 3 dpi. Despite the mortality observed in the three groups, NCT treatment prolongs the survival and suppresses viral replication to a greater extent than compared to the placebo control. For the delayed treatment reflecting a real-life situation, the efficacy is limited indicating that early administration is essential in the case of an HPAI outbreak.

Example 13. Prophylaxis of chickens challenged by HPAI Influenza A/Cattle/Texas/2024 (H5N1) virus with 1% NCT.

Five chickens inoculated with HPAI Influenza A/Cattle/Texas/2024 (H5N1) virus are placed 1 dpi in one cage with five contact chickens. A first control group of untreated inoculated and contact chickens become infected, and all inoculated and four contact chickens die.

A second group of inoculated and contact chickens are treated prophylactically with 1 ml each of aqueous 1% NCT solution aerosolized coarse spray over the whole bird (head and body) using an industry-standard commercial sprayer three times daily from 1 day before inoculation up to 5 dpi. Spray-deposited particles make their way into the respiratory tract by being deposited directly on the eye surface, internalized via the nares or oral cavity, or internalized indirectly as a consequence of normal bird behavior (e.g., preening). Although NCT prophylaxis does not prevent tracheal infection of the inoculated chickens, none has an infected cloaca, and only one dies. Only after stopping treatment do three contact chickens become positive, suggesting 1% NCT prophylaxis limits to large extent a severe outcome and chicken-to-chicken transmission of HPAI virus within or after the treatment period.

Example 14. Treatment of Influenza A (H5N1) dairy farm worker with 1% NCT eye drops and nasal/throat spray, and 1% NCT nasal/throat spray post exposure prophylaxis of household contacts.

Adult dairy farm worker has onset of redness and discomfort in the right eye. On presentation, subconjunctival hemorrhage and thin, serious drainage are noted in the right eye. The worker reports no contact with sick or dead wild birds, poultry, or other animals but reports direct and close exposure to dairy cows that appear to be well and with sick cows that show signs of illness as cows at other dairy farms in the same area with confirmed HPAI A (H5N1) virus infection (e.g., decreased milk production, lethargy, fever, and dehydration). The worker reports wearing gloves when working with cows but does not use any respiratory or eye protection.

Conjunctival and nasopharyngeal swab specimens are obtained from the right eye for influenza testing. The PCR results confirm influenza A (H5N1) in both specimens. The worker begins taking 1% NCT therapy (5× daily), i.e., eye drops (1×(0.1 ml/drop) per eye), nasal spray (3×(0.1 ml/puff) per nostril) and throat spray (5× puffs (0.1 ml/puff)) in the back of the mouth to the throat. Worker's household contacts use 1% NCT nasal/throat spray therapy (2-3× daily) as a post exposure prophylaxis for the duration, and they remain negative without developing any symptoms.

The next day, the worker reports no symptoms in both eyes; re-evaluation reveals subconjunctival hemorrhage in both eyes, with no visual impairment. Over subsequent days, the workers reports resolution of conjunctivitis without respiratory symptoms, and household contacts remain well.

Example 15. Treatment of Influenza A (H5N1) poultry farm workers with 1% NCT eye drops and nasal/throat spray.

Poultry in two commercial egg-layer operations are confirmed to have highly pathogenic avian influenza (HPAI) A (H5N1). The facility hires approximately 663 contract workers to conduct depopulation (i.e., euthanasia) of all poultry on the premises; 109 workers report symptoms and consent to testing. Among those who receive testing, nine receive a positive influenza A (H5N1) virus test result, and 19 receive a positive SARS-COV-2 test result. All nine workers who receive positive influenza A (H5N1) test results have conjunctivitis, and six report subjective fever or chills. Respiratory symptoms such as sore throat, cough, and shortness of breath are less frequently reported. Symptom onset occurs a median of 1 day after initial occupational exposure (range=1-8 days). All nine positive influenza A (H5N1) workers receive 1% NCT therapy (5× daily), i.e., eye drops (1×(0.1 ml/drop) per eye), nasal spray (3×(0.1 ml/puff) per nostril) and throat spray (5× puffs (0.1 ml/puff)) in the back of the mouth to the throat. Symptoms resolve for seven of the workers a median of 1 day after the start of NCT therapy, and the other two workers report ongoing or improving conjunctivitis two days after the start of NCT therapy.

The forgoing examples are illustrative only, and not meant to limit the scope of the inventions claimed herein.

What is claimed is:

1. A method of treating or preventing a viral infection in an animal, said method comprising administering a therapeutically effective amount of N-chlorotaurine, wherein the viral infection is selected from the group consisting of a coronavirus, influenza A, and respiratory syncytial virus (RSV).

2. The method according to claim 1, wherein said therapeutically effective amount of N-chlorotaurine comprises about a 0.1% to 2% solution of N-chlorotaurine.

3. The method according to claim 2, wherein said therapeutically effective amount of N-chlorotaurine comprises about a 1.0% solution of N-chlorotaurine in water.

4. The method according to claim 3, wherein said administering a therapeutically effective amount of N-chlorotaurine is selected from a group consisting of eye drop, aerosolized spray, inhalation, and intramammary instillation.

5. The method according to claim 4, wherein said coronavirus comprises severe acute respiratory syndrome coronavirus 2 (SARS-COV-2), or variants thereof, and said influenza A is selected from the group consisting of a H1N1, H3N2 and H5N1 influenza A virus strains.

6. The method according to claim 5, wherein said animal is a human.

7. The method according to claim 6, wherein said administering a therapeutically effective amount of N-chlorotaurine comprises using an eye drop to the eyes, and an aerosolized spray to the nostrils/throat.

8. The method according to claim 7, wherein said therapeutically effective amount of N-chlorotaurine comprises a dosage of about 0.1 ml to 0.2 ml to each of said eyes, about 0.1 ml to 0.3 ml to each of said nostrils, and about 0.3 ml to 0.6 ml to said throat, administered about 2 to 5 times per day.

9. The method according to claim 5, wherein said animal is a domesticated animal selected from the group consisting of a cow, goat, and sheep.

10. The method according to claim 9, wherein said instillation comprises using a cannula through a teat of an udder into a connected cavity of the mammary gland.

11. The method according to claim 10, wherein said therapeutically effective amount of N-chlorotaurine comprises a dosage of about 50 ml to 100 ml per administration to said mammary gland, administered about 2 times per day.

12. The method according to claim 5, wherein said animal is a bird.

13. The method according to claim 12, wherein said bird is domesticated poultry selected from the group consisting of a chicken, turkey, duck, and goose.

14. The method according to claim 13, wherein said aerosolized spray comprises using a coarse spray over the whole bird (head and body).

15. The method according to claim 14, wherein said therapeutically effective amount of N-chlorotaurine comprises a dosage of about 0.5 mg/Kg per administration to said whole bird, administered about 2 to 4 times per day.

16. The method according to claim 13, wherein said eye drop comprises using a pipette.

17. The method according to claim 16, wherein said therapeutically effective amount of N-chlorotaurine comprises a dosage of about 10 μl to 20 μl to each of said eyes, administered about 2 to 4 times per day.
